(19) United States
(12) Patent Application Publication
(10) Pub. No.: US 2007/0098802 A1
(43) Pub. Date: May 3, 2007

(54) Organic nanoparticles and associated methods

(76) Inventors: Isaac Farr, Corvallis, OR (US); Julio Cartagena, Isabela, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/263,725

(22) Filed: Oct. 31, 2005

(51) Int. Cl. A61K 9/14; A61K 38/13; A61K 31/7048; A61K 31/7034; A61K 31/573; A61K 31/43; A61K 31/56; A61K 31/337; A61K 31/401

(52) U.S. Cl. 424/489; 977/906; 514/179; 514/592; 514/11; 514/28; 514/192; 514/200; 514/449; 514/423

(57) ABSTRACT

Methods of preparing organic nanoparticles are provided. Such methods can include generating a mixture of an organic material, a first liquid, and a second liquid, wherein the organic material is more soluble in the second liquid than in the first liquid. The methods can also include adding a third liquid to the mixture which causes the mixture to form an emulsion. Such an emulsion can have a continuous phase including the first liquid and a discontinuous phase including the organic material and the second liquid. The organic material can be precipitated to form organic nanoparticles and the second liquid can diffuse into the continuous phase.

### BACKGROUND OF THE INVENTION

The production and use of small organic particles may improve the functionality of many substances, including drugs, dyes, pigments, etc. As the size of a collection of solid or semisolid particles is decreased, the exposed surface area of the material from which the particles are generated is greatly increased. Such an increase in surface area may be beneficial when utilizing solid particles in chemical reactions, reacting dyes or pigments with other ink components or recording media, increasing the bioavailability of drugs, etc.

As an example, many drugs are not readily taken up by a physiological system due to various factors such as hydrophobicity. For example, drugs such as various steroids, cyclosporine, and glyburide present delivery challenges due to their poor aqueous solubility and slow dissolution rate. Such low solubility may often result in low bioavailability, particularly given limited transit times through the gastrointestinal tract. Several approaches have been utilized in an attempt to enhance the in-vivo performance of many drugs, including micronization of the drug and emulsions containing the drug in a liquid form. These approaches, however, suffer from several disadvantages.

Micronization processes typically produce relatively large particles on the order of tens of microns to millimeters. Solid particles within this approximate size range may also be produced by precipitation, crystallization, lyophilization, or other forms of drying. For many pharmaceutical applications, particles of such size are sufficient. For some drugs, however, smaller sizes are desirable in order to increase the effective surface area available for dissolution, and to provide particles small enough to be taken up in the gastrointestinal tract. Producing drug particles of such small sizes often presents processing and stability challenges, particularly for formulations approaching the nanoparticle size range.

### BRIEF DESCRIPTION OF THE DRAWING

FIG. 1 is a graphical representation of organic nanoparticle size as a function of solute concentration in accordance with an exemplary embodiment of the present invention.

### DETAILED DESCRIPTION OF VARIOUS EMBODIMENTS

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a variable” includes reference to one or more of such variables.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. The active agent may be a therapeutic, a prophylactic, or a diagnostic agent. It is to be understood that the term “drug” is expressly encompassed by the present definition as many drugs and prodrugs are known to have specific physiologic activities. These terms of art are well-known in the pharmaceutical and medicinal arts.

As used herein “prodrug” refers to a molecule that will convert into a drug (its commonly known pharmacological active form). Prodrugs themselves can also be pharmacologically active, and therefore are also expressly included within the definition of an “active agent” as recited above.

The term “non-polymeric” refers to monomers and oligomers having a molecular weight less than about 3,000 Daltons.

Aspects of the present invention are directed towards the formation of organic nanoparticles from an organic material in a liquid mixture. It is intended that the scope of the claims of the present invention include any organic material that can be formed into nanoparticles in such a liquid mixture. Such organic materials include pharmaceutical compounds as well as non-pharmaceutical compounds.

According to one aspect of the present invention, a method of preparing organic nanoparticles is provided. Such a method can include generating a mixture of an organic material, a first liquid, and a second liquid, wherein the organic material is more soluble in the second liquid than in the first liquid. The method can also include adding a third liquid to the mixture which causes the mixture to form an emulsion. Such an emulsion can have a continuous phase including the first liquid and a discontinuous phase including the organic material and the second liquid. In one aspect, such an emulsion can be formed without surfactants or detergents. The organic material can be precipitated to form organic nanoparticles and the second liquid can diffuse into the continuous phase from the discontinuous phase.

Various relative degrees of solubility between the organic material, the first liquid, and the second liquid are contemplated, all of which are considered to be within the scope of the present invention. For example, in one aspect, the organic material can be more soluble in the second liquid than in the first liquid. In another aspect, the organic material can be at least substantially insoluble in the first liquid and at least substantially soluble in the second liquid. Regardless of the solubility of the organic material in the first and second liquids, these liquids can also exhibit various relative levels of miscibility with one another. In one aspect, however, the first liquid and the second liquid can form a solvent mixture that is at least substantially immiscible with respect to the third liquid. Additionally, in one aspect of the present invention, the organic material can be substantially insoluble in the third liquid.

In one aspect of the present invention, following the formation of the mixture of the organic material, the first liquid, and the second liquid, the third liquid can be added to form an emulsion. Alternatively, the mixture can be added to the third liquid to form an emulsion. It is contemplated that any order of mixing of the various components described herein that would result in the formation of an emulsion that would result in the creation of nanoparticles is included as part of the present invention. One effective non-limiting method of forming the emulsion includes titrating the third liquid into the mixture until the mixture turns cloudy, thus indicating the formation of the discontinuous phase of the emulsion.

Without intending to be limiting to a specific theory for the formation of the emulsion, the discontinuous phase may be formed because the organic material has associated with the second liquid due to its increased solubility therein. As such, due to insolubility of the organic material in the third liquid, the second liquid having the associated organic material has formed droplets that constitute the discontinuous phase. Depending on the various components utilized, the formation of the discontinuous phase of the emulsion can be facilitated by the immiscibility of the first and second liquid solvent mixture in the third liquid. This situation occurs for particular solvent mixtures that have been shown to form an emulsion in the absence of the organic material. For example, an emulsion will form when a mixture of ethanol and chloroform is titrated with water.

Though when described simplistically, the emulsion can have a continuous phase including the first liquid and a discontinuous phase including the organic material and the second liquid, in many cases the emulsion may be more complex. For example, the first and second liquids may not be completely partitioned into the continuous phase and the discontinuous phase respectively. In one aspect, the discontinuous phase may include a portion of the first liquid. In another aspect, the continuous phase may include a portion of the second liquid. Additionally, in one aspect a portion of the organic material can be present in the continuous phase. In another aspect, substantially all of the organic material can be present in the discontinuous phase.

Once the emulsion has been formed, the organic material may begin to precipitate to form organic nanoparticles. Without intending to be confined to any particular theory, it is believed that the organic material begins to precipitate along the interface between the discontinuous phase and the continuous phase due to the insolubility of the organic material in the third liquid. As the precipitate of the organic material begins to form, at least a portion of the second liquid may diffuse from the discontinuous phase into the continuous phase. Depending on the particular components involved, the organic nanoparticles may have a crystalline morphology that ranges from 0% to 100%. In other words, the crystalline morphology of the organic nanoparticles can range from purely crystalline to purely amorphous. In one aspect, the crystalline morphology of the organic nanoparticles can be at least substantially crystalline. In another aspect, the crystalline morphology of the organic nanoparticles can be at least substantially amorphous. In yet another aspect, the crystalline morphology of the organic nanoparticles can range from about 0% to about 100% crystalline. In a further aspect, the crystalline morphology of the organic nanoparticles can range from about 50% to about 100%. In yet a further aspect, the crystalline morphology of the organic nanoparticles can range from about 0% to about 50%.

It is possible that the organic material may not fully form into solidified nanoparticles, but may be of a jelly-like consistency. This may be due to the failure of the organic material to completely precipitate. Essentially complete precipitation can potentially be achieved by titrating additional third liquid into the mixture. In a situation such as this, it is believed that titration of an amount of the third liquid that is adequate to form an emulsion may not be adequate to allow the essentially complete precipitation of the organic material.

The resulting organic nanoparticles according to aspects of the present invention may be of any molecular configuration or size according to a particular desired use. In one aspect, the organic nanoparticles can be non-polymeric. In another aspect, the organic nanoparticles may be comprised of a precipitated small-molecule structure having a crystalline morphology as described above. Also, although any size of nanoparticle is contemplated, in one aspect, the organic nanoparticles can have an organic molecular weight from about 200 g/mol to about 3,000 g/mol. In another aspect, the organic nanoparticles can have an organic molecular weight from about 300 g/mol to about 1,500 g/mol.

Numerous first and second liquids can be utilized as components in various aspects of the present invention. Various liquids can be classified as either a first or a second liquid based on the relative solubility of an organic material therein. As such, a given liquid may be either a first or a second liquid, depending on the nature of the organic material utilized. It is intended that any two liquids to which the organic material exhibits differing solublities and which can form a suitable emulsion upon the addition of a third liquid be within the scope of the claims of the present invention. Though numerous first and second liquids are contemplated to be within the scope of the present invention, examples include, without limitation, alcohols, chlorinated solvents, ketones, and combinations thereof. Specific examples of useful alcohols include, without limitation, hexanol, pentanol, butanol, propanol, ethanol, methanol, and combinations thereof. Specific examples of useful chlorinated solvents include, without limitation, chloroform, methylene chloride, and combinations thereof. A specific example of a useful ketone can include, without limitation, acetone. As has been described, the combination of the first and second liquids depends on the organic material selected and its relative solubility in each liquid.

Various liquids can be utilized as the third liquid to facilitate the formation of the emulsion. In one aspect the organic material can be relatively insoluble in the third liquid, and in some aspects, the third liquid can be immiscible in the first and second liquid solvent mixture. Examples of suitable third liquids may include, without limitation, water, linear alkanes (C₅ to C₃₀), cycloalkanes (C₅ to C₈), and combinations thereof.

A broad range of organic materials may be formed into organic nanoparticles according to the various aspects of the present invention. Non-limiting examples of organic material can include therapeutically active agents, proteins, lipids, polysaccharides, proteoglycans, polynucleotides, waxes, dyes, pigments, anthracenes, stilbenes, p-terphenyls, perylene, phthalocyanine, pyrazoline, arylethynyl, poly(phenyleneethynylene)s, poly(phenylenevinylene)s, pentaphenylsilole, pseudoisocyanine derivatives, 1,2-bis-(4-methylbiphenyl)ethylene derivatives and combinations thereof, and combinations thereof.

Though benefit may be derived from forming nanoparticles from various types of organic materials, therapeutically active agents may be particularly beneficial when prepared according to embodiments of the present invention. Any active agent that is capable of being precipitated as a nanoparticle may be formed into organic nanoparticles by the methods of the present invention. Such active agents may be of small-molecule or macromolecular form, and may range from being non-soluble to completely soluble in bodily fluids. Examples of active agents that may benefit from the methods of the present invention include, without limitation, amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, derivatives, prodrugs, mixtures, and combinations thereof.

Following formation, the organic nanoparticles can be utilized as is, or they can be processed further. For example, in one aspect, the organic nanoparticles can be dried to at least substantially remove the first liquid, the second liquid, and the third liquid. Any process for removing the various liquids of the mixture from the organic nanoparticles that is known to one of ordinary skill in the art may be considered to be within the scope of the present invention. For example, such processes can include, without limitation, evaporation methods, centrifuging, cross-flow filtration, lypholization, etc. The nanoparticles can be utilized in their dried state, or they can be suspended in solution for utilization or storage.

When utilized in a liquid medium, various additional ingredients can be added to the suspension of organic nanoparticles. This may include nanoparticles that have been maintained in the three-liquid mixture or nanoparticles that have been re-suspended in a subsequent liquid medium. Any known ingredient can be added to the liquid medium, depending on the molecular make-up of the nanoparticles, the nature of the liquid medium, and/or the desired use of the resulting mixture. Alternatively, various additional ingredients can also be added to the organic nanoparticles in their isolated state. In either case, the additional ingredient can include surfactants, polymers, bioadhesive excipients, dispersants, biocidal agents, etc.

In one aspect, the organic nanoparticles may be coated with one or more surfactants, polymers, adhesion promoters, or other additives or excipients. They can also be incorporated into tablets, capsules, or other dosage forms, as described further herein. Various different excipients can be utilized in such active agent formulations. Examples include, without limitation, tableting aids, disintegrants, glidants, antioxidants and other preservatives, enteric coatings, taste masking agents, and the like. These excipients are well-known to those of ordinary skill in the art.

In one aspect, the organic nanoparticles can contain a surfactant to eliminate or reduce aggregation of the nanoparticles. Surfactants often adhere to the surface of the nanoparticles, and thus facilitate the dispersion of the nanoparticles in the mixtures in which the nanoparticles are formed, the medium in which the nanoparticles are taken up for administration, and the medium into which the particle is later delivered, such as the gastrointestinal fluid. Any known surfactant may be used in association with the nanoparticles. Suitable surfactants include small molecule surfactants and macromolecules such as polymeric surfactants. The surfactant may also contain a mixture of surfactants.

Various useful polymeric surfactants can include soluble and water-insoluble polymers, biodegradable and nonbiodegradable polymers, including hydrogels, thermoplastics, and homopolymers, copolymers and blends of natural and synthetic polymers. Representative polymers which can be used include hydrophilic polymers, such as those containing carboxylic groups, including polyacrylic acid. Bioerodible polymers including polyanhydrides, poly(hydroxy acids) and polyesters, as well as blends and copolymers thereof, also can be used. Representative bioerodible poly(hydroxy acids) and copolymers thereof which can be used include poly(lactic acid), poly(glycolic acid), poly(hydroxy-butyric acid), poly(hydroxyvaleric acid), poly(caprolactone), poly(lactide-co-caprolactone), and poly(lactide-co-glycolide). Polymers containing labile bonds, such as polyanhydrides and polyorthoesters, can be used optionally in a modified form with reduced hydrolytic reactivity. Positively charged hydrogels, such as chitosan, and thermoplastic polymers, such as polystyrene also can be used.

Various polymers, both natural and synthetic, can be added to the organic nanoparticles. Such polymers can be selected based on a variety of factors, such as polymer molecular weight, polymer hydrophilicity, polymer inherent viscosity, etc. Non-limiting examples of various polymers that may provide a benefit can include proteins, casein, gelatin, gluten, serum albumin, collagen, polysaccharides, polyphosphazenes, polyamides, polycarbonates, polyacrylamides, polysiloxanes, polyurethanes, celluloses, polymers of acrylic and methacrylic acids or esters, polyvinyl polymers, etc.

Wetting agents can also be utilized with the organic nanoparticles according to aspects of the present invention. Non-limiting examples of wetting agents include mannitol, dextrose, maltose, lactose, sucrose, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthlate, noncrystalline cellulose, magnesium aluminum silicate, triethanolamine, polyvinyl alcohol, and polyvinylpyrrolidone (PVP). One specific example of a useful wetting agent is Tyloxapol, which is a nonionic liquid polymer of the alkyl aryl polyether alcohol type.

Useful dispersants can include polyvinylpyrrolidone, polyethylene glycol, tyloxapol, poloxamers such as PLURONIC® F68, F127, and F108, which are block copolymers of ethylene oxide and propylene oxide, and polyxamines such as TETRONIC® 908 (POLOXAMINE® 908), which is a tetrafunctional block copolymer derived from sequential addition of propylene oxide and ethylene oxide to ethylenediamine, dextran, lecithin, dialkylesters of sodium sulfosuccinic acid such as AEROSOL® OT, which is a dioctyl ester of sodium sulfosuccinic acid, DUPONOL® P, which is a sodium lauryl sulfate, TRITON® X-200, which is an alkyl aryl polyether sulfonate, TWEEN® 20 and TWEEN® 80, which are polyoxyethylene sorbitan fatty acid esters, Carbowax 3550 and 934, which are polyethylene glycols, Crodesta F-110, which is a mixture of sucrose stearate and sucrose distearate, and Crodesta SL-40, and SA90HCO, which is C₁₈H₃₇CH₂(CON(CH₃)CH₂(CHOH)₄CH₂OH)2.

Various pharmaceutical uses for the resulting organic nanoparticles, whether in a dried or liquid state, can be desireable. Any medical or veterinary condition that can be treated with a pharmaceutical can benefit from implementation of the present invention. As such, pharmaceutical organic nanoparticles can be delivered through a variety of dosage forms. Such dosage forms may include, without limitation, oral formulations including solids and liquids, transdermal formulations including adhesive matrix patches, liquid reservoir patches, iontophoretic patches, creams, ointments, gels, etc., aerosol and liquid spray formulations, parenteral formulations, sublingual formulations, rectal and vaginal formulations, implanted or injected depot formulations, etc.

### EXAMPLE

A 0.5% (w/v) mixture of 5 mg/ml of glyburide in 70% ethanol and 30% chloroform by weight is prepared. To this mixture, water is added until clouding is observed at which point the emulsion has formed. Light scattering and scanning electron microscopy show the resulting nanoparticle size is on average about 500 nm. A few large nanoparticles may be observed of up to 1 to 3 μm.

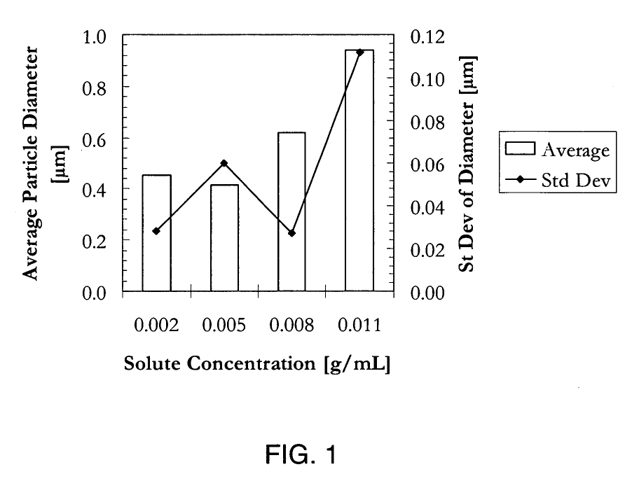

FIG. 1 shows the ability to control the final particle size based on the initial concentration in the ethanol/chloroform solution. The data from FIG. 1 was obtained by dissolving either 0.002 g/ml, 0.005 g/ml, 0.008 g/ml, or 0.011 g/ml of glyburide in a solution of 80:20 ethanol: chloroform solution. To this, water was added until the cloud point was reached. Following precipitation of the glyburide, SEM's were used to measure the resulting particle size.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications may be made without departing from the principles and concepts set forth herein.

What is claimed is:

1. A method of preparing organic nanoparticles, comprising:
generating a mixture of an organic material, a first liquid, and a second liquid, wherein the organic material is more soluble in the second liquid than in the first liquid;
adding a third liquid to the mixture which causes the mixture to form an emulsion, said emulsion having a continuous phase including the first liquid and a discontinuous phase including the organic material and the second liquid; and
precipitating the organic material to form organic nanoparticles, wherein at least a portion of the second liquid diffuses from the discontinuous phase to the continuous phase.

2. The method of claim 1, wherein the organic nanoparticles have an organic molecular weight from about 200 g/mol to about 3,000 g/mol.

3. The method of claim 1, wherein the organic nanoparticles have an organic molecular weight from about 300 g/mol to about 1,500 g/mol.

4. The method of claim 1, wherein the organic material is selected from therapeutically active agents, proteins, lipids, polysaccharides, proteoglycans, polynucleotides, waxes, dyes, pigments, anthracenes, stilbenes, p-terphenyls, perylene, phthalocyanine, pyrazoline, arylethynyl, poly(phenyleneethynylene)s, poly(phenylenevinylene)s, pentaphenylsilole, pseudoisocyanine derivatives, 1,2-bis-(4-methylbiphenyl)ethylene derivatives and combinations thereof.

5. The method of claim 1, wherein the organic material is selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfioquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

6. The method of claim 5, wherein the organic material is glyburide.

7. The method of claim 5, wherein the organic material is prednisolone.

8. The method of claim 5, wherein the organic material is glipizide.

9. The method of claim 1, wherein the first liquid and second liquid are selected from alcohols, chlorinated solvents, ketones, and combinations thereof.

10. The method of claim 9, wherein at least one of the first liquid and the second liquid is an alcohol selected from hexanol, pentanol, butanol, propanol, ethanol, methanol and combinations thereof.

11. The method of claim 9, wherein at least one of the first liquid and the second liquid is a chlorinated solvent selected from chloroform, methylene chloride, and combinations thereof.

12. The method of claim 9, wherein at least one of the first liquid and the second liquid is acetone.

13. The method of claim 1, wherein the first and second liquid include respective combinations selected from an alcohol and a chlorinated solvent, an alcohol and a ketone, a chlorinated solvent and a ketone, a chlorinated solvent and an alcohol, a ketone and an alcohol, a ketone and a chlorinated solvent, and combinations thereof.

14. The method of claim 1, wherein the first liquid and the second liquid form a solvent mixture that is at least substantially immiscible with respect to the third liquid.

15. The method of claim 1, wherein the third liquid is a member selected from water, linear alkanes (C₅ to C₃₀), cycloalkanes (C₅ to C₈), and combinations thereof.

16. The method of claim 1, wherein the discontinuous phase includes a portion of the first liquid.

17. The method of claim 1, wherein the continuous phase includes a portion of the second liquid.

18. The method of claim 1, wherein the organic nanoparticles are non-polymeric.

19. The method of claim 1, further comprising adding at least one additional ingredient following the precipitation of the organic nanoparticles.

20. The method of claim 19, wherein the additional ingredient is a member selected from surfactants, polymers, bioadhesive excipients, dispersants, biocidal agents, and combinations thereof.

21. The method of claim 1, wherein the organic material is at least substantially insoluble in the first liquid, and wherein the organic material is at least substantially soluble in the second liquid.

22. The method of claim 1, further comprising drying the organic nanoparticles to at least substantially remove the first liquid, the second liquid, and the third liquid.

23. The method of claim 1, further comprising:
a preliminary step of determining a desired organic nanoparticle size; and
generating the mixture having a sufficient concentration of the organic material to achieve the desired organic nanoparticle size.

24. A plurality of organic nanoparticles prepared according to the method of claim 22.

25. A plurality of organic nanoparticles prepared according to the method of claim 1.

26. A method of preparing organic nanoparticles, comprising:
means for generating a mixture of an organic material, a first liquid, and a second liquid, wherein the organic material is more soluble in the second liquid than in the first liquid;
means for forming an emulsion, said emulsion having a continuous phase including the first liquid and a discontinuous phase including the organic material and the second liquid; and
means for precipitating the organic material to form organic nanoparticles, wherein at least a portion of the second liquid diffuses from the discontinuous phase to the continuous phase.
